(19) United States
(12) Patent Application Publication
(10) Pub. No.: US 2024/0144381 A1
(43) Pub. Date: May 2, 2024

(54) INTELLIGENT WORKFLOW FOR HEALTHCARE

(71) Applicant: INTERNATIONAL BUSINESS MACHINES CORPORATION, Armonk, NY (US)

(72) Inventors: Michael Jack MARTINE, Chapel Hill, NC (US); Sarah Diane GREEN, Chandler, AZ (US); David DRAEGER, Longwood, FL (US); Stan Kevin DALEY, Espanola, NM (US); Ira L. ALLEN, Dallas, TX (US); John Donald VASQUEZ, Munich (DE)

(21) Appl. No.: 18/051,988

(22) Filed: Nov. 2, 2022

(51) Int. Cl. G06Q 40/08; G16H 20/00; G16H 40/67

(52) U.S. Cl. CPC G06Q 40/08; G16H 20/00; G16H 40/67; G06Q 2220/00

(57) ABSTRACT

Device data is obtained from one or more devices for an individual. The one or more devices include one or more medical monitoring devices. Data is also obtained from one or more exogenous sources, and the data includes current data relating to one or more medical resources. The device data obtained from the one or more devices and the data from the one or more exogenous sources are analyzed using an artificial intelligence agent. Based on the analyzing, one or more recommendations are provided. The one or more recommendations include an indication of a device to use that is different from the one or more devices to obtain different device data for the individual.

### BACKGROUND

One or more aspects relate, in general, to dynamic processing within a computing environment, and in particular, to improving such processing, as it relates to healthcare.

Healthcare, over time, has become increasingly digital. As part of the consumerization of healthcare, data has become more prevalent and available to patients and providers at a rate that is unprecedented. This creates a problem in which the volume of information exceeds the capacity of the individual to understand what actions can be taken, what behaviors should be exhibited, and what decisions influence actions.

Information overload makes it difficult to identify and parse useful information from digital noise since one must make sense of volumes of aggregated data from multiple sources. Without individualized insights, it is difficult to use the data to improve health outcomes.

### SUMMARY

Shortcomings of the prior art are overcome, and additional advantages are provided through the provision of a computer-implemented method of dynamic processing within a computing environment. The computer-implemented method includes obtaining device data from one or more devices for an individual. The one or more devices include one or more medical monitoring devices. Data is obtained from one or more exogenous sources, and the data includes current data relating to one or more medical resources. The device data obtained from the one or more devices and the data from the one or more exogenous sources are analyzed using an artificial intelligence agent. Based on the analyzing, one or more recommendations are provided. The one or more recommendations include an indication of a device to use that is different from the one or more devices to obtain different device data for the individual.

Computer systems and computer program products relating to one or more aspects are also described and may be claimed herein. Further, services relating to one or more aspects are also described and may be claimed herein.

Additional features and advantages are realized through the techniques described herein. Other embodiments and aspects are described in detail herein and are considered a part of the claimed aspects.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 depicts one example of a computing environment to perform, include and/or use one or more aspects of the present invention;

FIG. 2A depicts one example of sub-modules of an intelligent workflow for healthcare module of FIG. 1, in accordance with one or more aspects of the present invention;

FIG. 2B depicts one example of a set-up workflow sub-module of FIG. 2A, in accordance with one or more aspects of the present invention;

FIG. 2C depicts one example of an execution of workflow sub-module of FIG. 2A, in accordance with one or more aspects of the present invention;

FIG. 3A depicts one example of processing related to an intelligent workflow for healthcare, in accordance with one or more aspects of the present invention;

FIG. 3B depicts one example of processing of an artificial intelligence agent initiated by the intelligent workflow of FIG. 3A, in accordance with one or more aspects of the present invention;

FIG. 4 depicts one example of capabilities of an intelligent workflow for healthcare, in accordance with one or more aspects of the present invention; and

FIG. 5 depicts one example of a machine learning training system used in accordance with one or more aspects of the present invention.

### DETAILED DESCRIPTION

In one or more aspects, a capability is provided to perform intelligent workflow. Intelligent workflow is the orchestration of automation, artificial intelligence, analytics, and skills to fundamentally change how work is performed. In one or more aspects, intelligent workflow is defined and used for healthcare. As applied to healthcare, in one or more aspects, the intelligent workflow collects data, makes sense of the vast amount of available data, makes the right connections between the data, and suggests what additional data may be used to make one or more recommendations for next best actions to improve outcomes, such as health outcomes for individuals.

In one or more aspects, the intelligent workflow augments an individual's access to volumes of information in a way that protects the individual's confidential data while providing the individual with options and comprehensible information to make informed decisions. The intelligent workflow addresses the fact that individuals are different.

In one or more aspects, dynamic temporal control of data is placed in the hands of an individual subscribing to the intelligent workflow. The control is provided via, e.g., one or more smart contracts established by the individual. A smart contract is, for instance, a computer program or a transaction protocol that is intended to automatically execute, control or document legally relevant events and actions according to the terms of a contract or agreement. In one example, the terms of the contract or agreement relate to, e.g., medical data, conditions, health concerns and/or aspects of an individual. The smart contract explicitly indicates by the individual what information may be disclosed, to whom, and under what circumstances.

In one or more aspects, the intelligent workflow initiates a user-authorized intelligent workflow health agent (e.g., an artificial intelligence agent) that collects data from dedicated sensors of the individual, environmental monitors (e.g., thermometers, wind sensors, humidity sensors, etc.), security appliances, cameras, etc., and/or exogenous sources (e.g., medical journals, governmental rulings, device manufacturer literature, etc.); analyzes the data; detects changes in an individual's condition across data from multiple sources; recommends additional resources, e.g., devices to be used by an individual to improve data and/or quantify improvements; connects with commerce engines to facilitate obtaining the devices; suggests practitioners and/or services to the individual; takes action for an individual based on the smart contract, if, for instance, a certain predefined condition occurs (e.g., incapacitation of the individual); and/or provides artificial intelligence-driven coordination of care at the edge (e.g., close to the individual).

One or more aspects of the present invention are incorporated in, performed and/or used by a computing environment. As examples, the computing environment may be of various architectures and of various types, including, but not limited to: personal computing, client-server, distributed, virtual, emulated, partitioned, non-partitioned, cloud-based, quantum, grid, time-sharing, cluster, peer-to-peer, wearable, mobile, having one node or multiple nodes, having one processor or multiple processors, and/or any other type of environment and/or configuration, etc. that is capable of executing a process (or multiple processes) that, e.g., performs intelligent workflow and/or performs one or more other aspects of the present invention. Aspects of the present invention are not limited to a particular architecture or environment.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

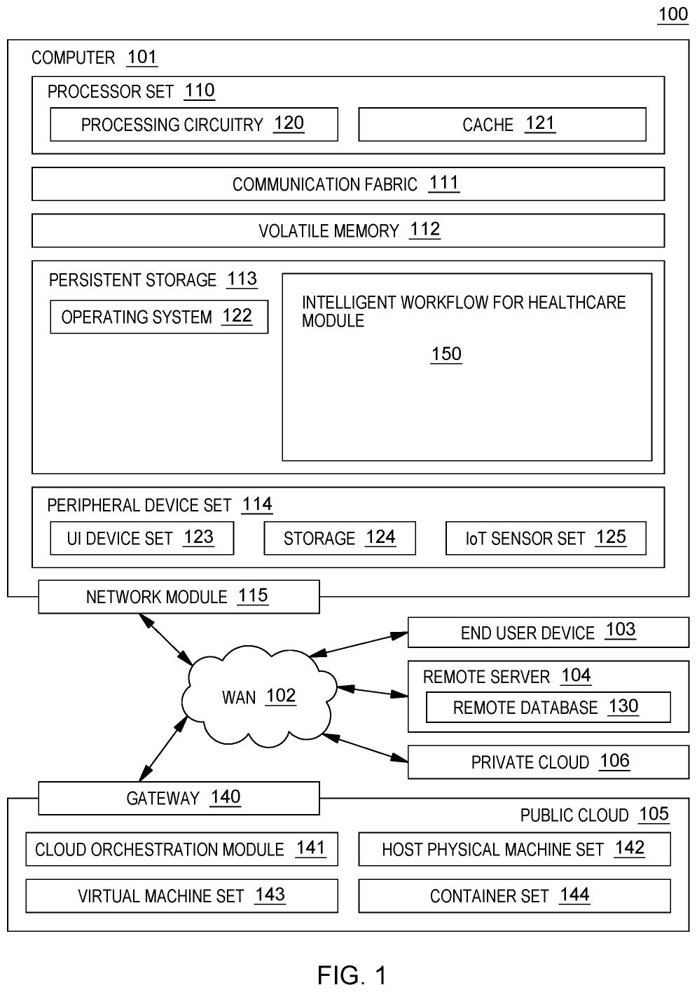

One example of a computing environment to perform, incorporate and/or use one or more aspects of the present invention is described with reference to FIG. 1. In one example, a computing environment 100 contains an example of an environment for the execution of at least some of the computer code involved in performing the inventive methods, such as intelligent workflow for healthcare code or module 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

Communication fabric 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 150 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The computing environment described above is only one example of a computing environment to incorporate, perform and/or use one or more aspects of the present invention. Other examples are possible. For instance, in one or more embodiments, one or more of the components/modules of FIG. 1 are not included in the computing environment and/or are not used for one or more aspects of the present invention. Further, in one or more embodiments, additional and/or other components/modules may be used. Other variations are possible.

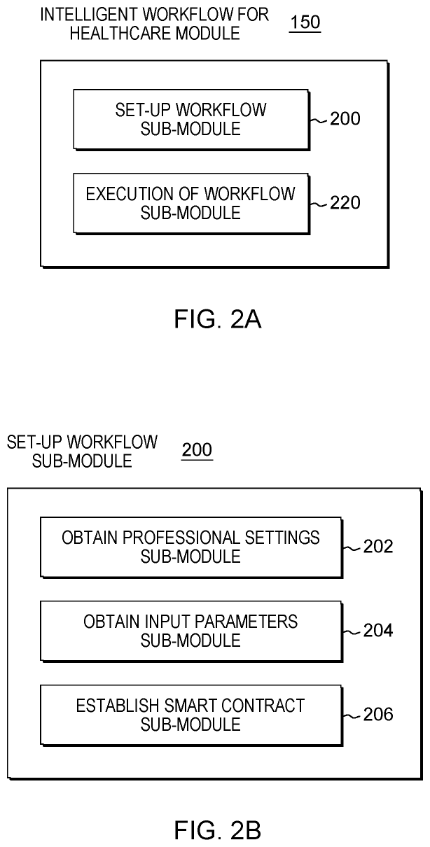

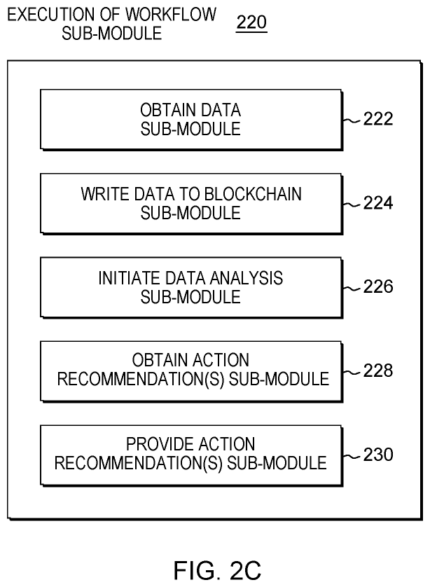

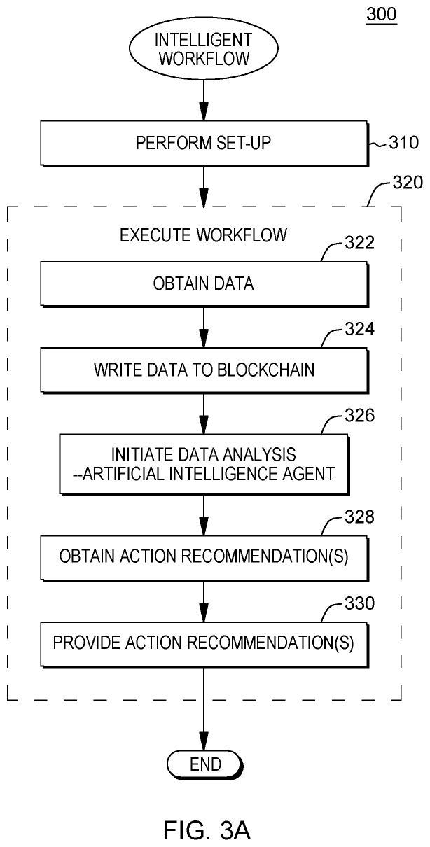

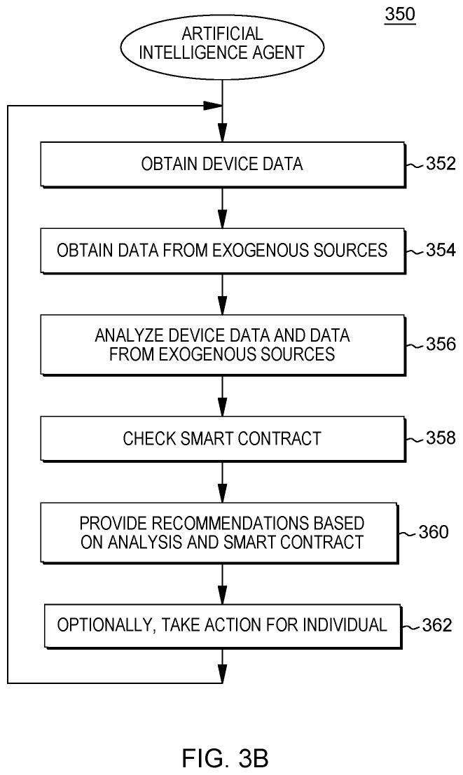

Further details relating to intelligent workflow for healthcare are described with reference to FIGS. 2A-3B. FIGS. 2A-2C depict further details of an intelligent workflow for healthcare module (e.g., intelligent workflow for healthcare module 150 of FIG. 1) that includes code or instructions used to perform intelligent workflow, in accordance with one or more aspects of the present invention, and FIGS. 3A-3B depict one embodiment of a process to perform intelligent workflow, in accordance with one or more aspects of the present invention.

In one or more aspects, referring to FIG. 2A, an intelligent workflow for healthcare module (e.g., intelligent workflow for healthcare module 150) includes, in one example, various sub-modules to be used to perform intelligent workflow. The sub-modules are, e.g., computer readable program code (e.g., instructions) in computer readable media, e.g., persistent storage (e.g., persistent storage 113, such as a disk) and/or a cache (e.g., cache 121), as examples. The computer readable media may be part of a computer program product and may be executed by and/or using one or more computers, such as computer(s) 101, as an example; processors, such as a processor of processor set 110; and/or processing circuitry, such as processing circuitry of processor set 110, etc. Additional and/or other computers, processors and/or processing circuitry may be used to execute one or more of the sub-modules and/or portions thereof. Many examples are possible.

Example sub-modules of intelligent workflow for healthcare module 150 include, for instance, a set-up workflow sub-module 200 used to perform set-up for the intelligent workflow, and an execution of workflow sub-module 220 used to perform execution of the intelligent workflow. Further details relating to set-up workflow sub-module 200 are described with reference to FIG. 2B and further details relating to execution of workflow sub-module 220 are described with reference to FIG. 2C. Although various sub-modules are described, an intelligent workflow for healthcare module, such as intelligent workflow for healthcare module 150, may include additional, fewer and/or different sub-modules. A particular sub-module may include additional code, including code of other sub-modules, less code, and/or different code. Further, additional and/or other modules may be used to perform intelligent workflow. Many variations are possible.

Referring to FIG. 2B, in one example, set-up workflow sub-module 200 includes an obtain professional settings sub-module 202 to obtain selected individualized settings (e.g., baselines and/or thresholds for an individual) from one or more health professionals, such as a physician, physician assistant, nurse, etc.; an obtain input parameters sub-module 204 to obtain, for instance, device specifications from one or more device manufacturers of one or more devices to be used by an individual; and an establish smart contract sub-module 206 to establish a smart contract for an individual. In one example, the terms of the contract relate to, e.g., medical data, conditions and/or health concerns and/or aspects of the individual.

Referring to FIG. 2C, in one example, execution of workflow sub-module 220 includes an obtain data sub-module 222 to be used to obtain data from one or more sources, such as one or more devices monitoring the individual, one or more environmental devices, security devices, cameras, and/or other devices that may provide information for the individual, and/or other data. Execution of workflow sub-module 220 further includes, for instance, a write data to blockchain sub-module 224 to be used to write the obtained data (or at least a portion of it—e.g., confidential and/or sensitive data of the individual) to a blockchain or other security mechanism. A blockchain is, for example, a shared, immutable ledger that includes a growing list of records, called blocks, that are securely linked. The blockchain maintains security and confidentiality of an individual's data. Although in the examples herein, blockchain is used as the security mechanism, other security mechanisms may be used. Blockchain is only one example.

Execution of workflow sub-module 220 further includes, in one example, an initiate data analysis sub-module 226 to be used to initiate data analysis of the obtained data. In one example, it includes initiating an artificial intelligence agent to be used to obtain data, perform analysis and provide recommendations. Execution of workflow sub-module 220 further includes, for instance, an obtain action recommendation(s) sub-module 228 to be used to obtain one or more recommendations based on the analysis; and a provide action recommendation(s) sub-module 230 to be used to provide the one or more action recommendations to one or more entities, such as the individual, a provider (e.g., a health professional—e.g., physician, physician assistant, nurse, specialist, pharmacist, etc.), other providers (e.g., service providers—e.g., those that provide food service, insurance, care givers, etc.), etc. depending on authorization provided in one or more smart contracts. Although example professionals, service providers, providers, entities, etc. are indicated herein, additional, fewer and/or other professionals, service providers, providers and/or entities may be specified. Those indicated herein are just examples.

The sub-modules are used, in accordance with one or more aspects of the present invention, to perform intelligent workflow processing, as further described with reference to FIGS. 3A-3B. In one example, FIG. 3A depicts one example of an intelligent workflow process, and FIG. 3B depicts one example of processing of an artificial intelligence agent initiated by the intelligent workflow process of FIG. 3A, in accordance with one or more aspects of the present invention. The intelligent workflow process is executed, in one or more examples, by a computer (e.g., computer 101, other computer(s) or server(s), etc.), and/or a processor or processing circuitry (e.g., of processor set 110 or other processor sets). Further, the artificial intelligence agent is executed, in one or more examples, by a computer (e.g., computer 101, remote server 104, in public cloud 105, in private cloud 106, other computer(s) or server(s), etc.), and/or a processor or processing circuitry (e.g., of processor set 110 or other processor sets). Although example computers, servers, processors and/or processing circuitry are provided as examples, additional, fewer and/or other computers, servers, processors and/or processing circuitry may be used for the intelligent workflow process and/or the artificial intelligence agent. In one example, code or instructions implementing the intelligent workflow process and the artificial intelligence agent are part of a module, such as module 150. In other examples, the code may be included in one or more modules and/or in one or more sub-modules of the one or more modules. For instance, the code for the intelligent workflow process and the artificial intelligence agent may be combined in one module, be in separate modules and/or in one or more modules. The one or more modules may be in persistent storage (e.g., persistent storage 113 or other persistent storage), cache (e.g., cache 121 or other cache), and/or other storage. Various options are available.

Referring to FIG. 3A, as one example, an intelligent workflow process 300 performs 310 set-up for use in intelligent workflow processing. In one example, the set-up includes establishing, by an individual that would like to benefit from intelligent workflow processing, at least one smart contract that explicitly indicates what information may be disclosed, to whom, and under what circumstances. This is an explicit act performed by the individual. It is not an opt-in; instead, it is explicit permission via one or more smart contracts. The smart contract defines the explicit permissions provided to the intelligent workflow and specifically, to the artificial intelligence agent, regarding the individual's data. The individual's data is secured by use of blockchain (or other security mechanism), to which the individual's health provider subscribes, providing an intelligent workflow.

In one or more aspects, a user-authorized smart contract may be set up to enable action to be taken on the individual's behalf if the individual is incapacitated (or based on another predefined situation) and cannot make a decision directly to share the data. For example, if the individual's blood sugar drops below a safe threshold, the artificial intelligence agent may reach out to medical support for the individual and contact a designated person. As the artificial intelligence agent learns more about the individual, the agent may prompt the individual with options to adjust the smart contract given a multitude of other data, such as, for instance, geolocation, accelerometer, weather, heart rate exceeds a threshold, etc. Many types of data and/or options are possible.

In one example, the smart contract prepared for an individual is adjustable and one or more provisions may be added, deleted, modified and/or revoked over time. An adjustment to the smart contract may be permanent (i.e., until adjusted again) or temporary in which it is for a selected time period. Many variations are possible.

In one example, the set-up further includes obtaining professional (e.g., physician, physician assistant, nurse, etc.) settings (e.g., baseline levels) for an individual. For instance, the professional may indicate that a baseline glucose level for the individual should be X and/or that a particular device, such as a glucose monitor, should be set at a particular setting for an individual. Similar settings and baselines may be provided for other conditions, including, for instance, blood pressure, heart rate, for devices inserted within the individual, such as an implantable defibrillator or pace maker, etc. Settings and/or baselines for many conditions and/or devices used by the individual may be provided. Those provided, however, would be authorized by the individual in one or more smart contracts.

The professional may also specify, for instance, one or more thresholds that indicate an alert is to be signaled if a particular level (e.g., glucose level, heart rate, pulse, defibrillator reading, etc.) has a predetermined relationship with the threshold (e.g., reaches the threshold, exceeds the threshold, etc.). Although one or more settings/thresholds are specified as examples, additional, fewer and/or other settings and/or thresholds may be used. Further settings and/or thresholds may be specified. Many examples are possible.

In one example, the set-up also includes obtaining input parameters/specifications relating to one or more devices (e.g., sensors, monitors, etc.) used by an individual. For instance, a manufacturer may indicate that a particular device, such as a glucose monitor, should be set at a particular setting or within a particular range. Similar input parameters/specifications may be provided for other monitoring devices, including those that monitor, for instance, blood pressure, heart rate, for devices inserted within the individual, such as an implantable defibrillator or pace maker, etc. Parameters/specifications for many devices may be provided. The parameters/specifications may be generic for the devices and/or may be more specific depending on an individual's condition. If it is more specific, then individual authorization is to be provided by the individual in, e.g., one or more smart contracts. Although one or more parameters/specifications are indicated as examples, additional, fewer and/or other parameters and/or specifications may be used. Many examples are possible.

Subsequent to performing the set-up, intelligent workflow process 300 executes 320 the workflow (e.g., tasks, actions, steps to perform intelligent workflow processing). In one example, intelligent workflow process 300 obtains (e.g., receives, is sent, is provided, retrieves, etc.) 322 data. For instance, data is obtained that relates to the individual, such as the health of an individual. As an example, the devices indicated as part of the set-up and/or other devices are used to monitor and report data relating to the health of an individual. Example data includes, for instance, glucose readings, blood pressure readings, temperature, heart rate, pulse, environmental data, and/or physical activity, as just some examples. Although example devices and/or data are described herein, other devices and/or data may be used/obtained.

In one example, intelligent workflow process 300 writes 324 the obtained data to a security mechanism, such as a blockchain. The blockchain stores the data for secure sharing between the individual and other entities, e.g., a medical practitioner, in immutable form. Further, it enables artificial intelligence (e.g., an artificial intelligence agent) to wholistically analyze data, in totality, and make one or more next best action recommendations.

Further, in one example, intelligent workflow process 300 initiates 326 the data analysis. For example, intelligent workflow process 300 authorizes and initiates an artificial intelligence agent to perform analysis of the data stored in the blockchain. In one example, an artificial intelligence agent is, e.g., code, module(s), processing, etc. that perceives its environment, takes actions autonomously to achieve goals, and may improve its performance with learning or may use knowledge. The artificial intelligence agent may be part of intelligent workflow for healthcare module 150 or may be wholly or partially in one or more other modules in persistent storage (e.g., persistent storage 113) or other storage, in memory/storage of another computer, server and/or in the cloud, as examples.

In one example, the initiated artificial intelligence agent continuously (or periodically) monitors current conditions of the individual and compares those against one or more secure baselines (e.g., established during set-up). The artificial intelligence agent generates and provides unique insight to the individual relating to their next best action(s), using blockchain to store the data.

Based on the data analysis, intelligent workflow process 300 obtains 328 one or more action recommendations (also referred to as recommendations herein). The one or more action recommendations may include, for instance, a recommendation to use and/or obtain (e.g., purchase, lease, rent, borrow, etc.) one or more other devices (e.g., sensors, monitors, other devices) to monitor the individual or an environment relating to the individual (e.g., temperature or humidity within the home or area surrounding the individual). These other devices may be used in addition to or in place of one or more other devices used by the individual. The other devices collect additional, less and/or different data to provide a higher certainty of recommendation (e.g., use of glucose strips vs active glucose monitoring, both accurate, however one's situation might warrant more frequent and NFC (Near Field Communication)/RFID (Radio Frequency Identification) enabled monitoring for constant monitoring). Further, the one or more action recommendations may include a recommendation of a provider (e.g., a particular type of professional; a service provider, such as one that provides, e.g., nutritional service, food service, care giver, insurance, and/or different types of service; and/or other providers, etc.); a recommendation to obtain additional data (e.g., by a subscription or purchase); etc. Many examples are possible.

Based on obtaining the one or more action recommendations, intelligent workflow process 300 provides 330 the obtained action recommendations to one or more entities. For instance, the one or more action recommendations may be provided to the individual (e.g., at end user device 103 or another device) or another entity (e.g., another person, a provider (e.g., a health professional, a service provider, etc.), a company, etc.) explicitly authorized by the individual via, e.g., one or more smart contracts, to receive the information. The one or more action recommendations may be provided as an alert to an individual to take action to improve wellness, for coordination of care, device purchase, etc. In one example, the action recommendations may be updated to the blockchain and/or the actions taken by the individual may be specified and updated to the blockchain. Other examples are possible.

Further details regarding data analysis using, e.g., an artificial intelligence agent, are described with reference to FIG. 3B. In one example, an artificial intelligence agent 350 obtains (e.g., receives, is sent, is provided, retrieves, etc.) 352 device data. For instance, device data is obtained that relates to the individual, such as the health of an individual. As an example, the device data is obtained from one or more devices used to monitor and report data relating to the health of an individual. Example data includes, for instance, glucose readings, blood pressure readings, temperature, heart rate, pulse, carbon dioxide, and/or physical activity, as just some examples. Further, in one or more examples, the device data is data output from other types of devices, such as environment devices associated with the individual, such as a thermostat, humidity monitor, smart doorbells, security devices, carbon monoxide detectors, cooking sensors, sensors that can observe and detect anomalies, etc. Although example devices and/or data are described herein, other devices and/or data may be used/obtained. The obtained device data (or at least a portion of the data—e.g., confidential and/or sensitive data) is saved, e.g., in a blockchain for security and confidentiality.

Further, in one example, artificial intelligence agent 350 obtains 354 data from exogenous or external sources (e.g., medical journals, manufacturer specifications, new Federal Drug Administration regulations and/or approved devices, environmental data from, e.g., the National Oceanic and Atmospheric Administration and/or other agencies, public health data from, e.g., the Centers for Disease Control and Prevention and/or other agencies, social media, news stories, geolocation matching, public safety, traffic patterns, etc.). For instance, the artificial intelligence agent obtains information regarding the latest medical devices, latest baselines for certain conditions, environmental data and/or other data, etc. This information may be obtained from one or more exogenous sources and/or the set-up. As an example, the artificial intelligence agent is able to review voluminous amounts of data in a timely manner to gain the most up-to-date information to be used in making recommendations. In one example, the device data and/or the data from exogenous sources is obtained at selected intervals (e.g., at periodic intervals, continuously, etc.).

In one example, the initiated artificial intelligence agent analyzes 356 the device data and the data from the exogenous sources. As part of analysis, in one example, the data is normalized to standardize the data being analyzed and to recognize and understand the relationships between the data. For instance, an understanding is obtained as to whether a certain environmental condition affects an individual's condition, whether an adjustment of one monitor affects another monitor, etc. In one example, the artificial intelligence agent continuously (or periodically) monitors current conditions and obtains up-to-date data to perform analysis and obtain the understanding to make recommendations.

In one example, the artificial intelligence agent obtains the data, analyzes it, and compares the analyzed data against one or more secure baselines of the individual (e.g., provided in set-up). The artificial intelligence agent generates and provides unique insight to the individual regarding their next best action(s), using blockchain to store the data. In one example, artificial intelligence agent 350 checks 358 the one or more smart contracts of the individual to determine the permissions authorized in the smart contract(s) by the individual. For instance, the artificial intelligence agent checks which one or more entities are authorized to receive information (e.g., medical information) of the individual; what types of information each authorized entity is allowed to receive; what, if any, actions may be taken on behalf of the individual, etc.

Based on the analysis and the check of the smart contract, artificial intelligence agent 350 provides 360 one or more recommendations for the individual. The one or more recommendations may include, for instance, the purchase or use of one or more additional devices; subscription or purchase of additional data from one or more sources; recommendation of additional/alternative sensors, monitors and/or other devices to collect additional data to have a higher certainty of recommendation (e.g., use of glucose strips vs active glucose monitoring); recommendation of additional providers (e.g., different types of health professionals, such as a specialist, therapist, etc.; service providers, such as those that provide nutritional services, food service, insurance services, care giver, etc.; and/or other providers). Many examples are possible.

In one or more examples, the one or more recommendations are provided to the intelligent workflow process (e.g., intelligent workflow process 300), which is responsible for alerting one or more entities of the recommendations, as specified by the smart contract. In another example, the one or more recommendations are provided to the one or more entities by the artificial intelligence agent. Various possibilities exist. For instance, one or more types of recommendations may be provided to the intelligent workflow process to be forwarded to the one or more entities, while other types of recommendations (e.g., more critical-as predefined) may be provided to the entities by the artificial intelligence agent. As another example, recommendations to one or more types of entities (e.g., individual, service provider) are provided using the intelligent workflow process, while recommendations to other types of entities (e.g., medical professional) are provided directly by the artificial intelligence agent. Again, many possibilities exist.

In one or more examples, the recommendations are automatically stored to the smart contract if the individual explicitly authorizes such changes to the smart contract. Further, in one or more examples, based on an individual choosing to accept the one or more recommendations, the accepted recommendations are included in the smart contract by the individual or automatically by the intelligent workflow, as the intelligent workflow learns of the changes, provided the intelligent workflow is authorized to make such changes.

In one example, artificial intelligence agent 350 may take action 362 on behalf of the individual in certain prescribed situations. For instance, the smart contract takes control for the individual when a certain pre-specified situation occurs (e.g., the individual is incapacitated). So, in some form, the individual remains in control, since the individual predetermines what is to happen if the situation occurs. For instance, if authorized by the smart contract and the individual becomes incapacitated, then the artificial intelligence agent 350 contacts one or more entities, as specified in the smart contract. Other examples are also possible.

An example of using an artificial intelligence agent (e.g., artificial intelligence agent 350) includes: Assume the artificial intelligence agent has data A & B and if artificial intelligence agent had data C, it could make a conclusion. C could be some other sensor data the artificial intelligence agent may recommend.

The artificial intelligence agent prompts the individual to purchase a device/sensor and/or subscribe to a data source, as examples, to obtain the desired data and facilitates saving that information to the individual's smart contract.

The artificial intelligence agent, in one aspect, facilitates submission to the individual's insurance to request the recommended device, providing justification and automation of the request to the insurance company.

Alternatively, the data may be available on a device the individual has but is not currently shared with the artificial intelligence agent. In this example, the artificial intelligence agent prompts the individual to grant access to the data, either temporarily or permanently via, e.g., a smart contract. Although various examples have been provided, there are many possibilities.

As described herein, the intelligent workflow is based on real, secure, confidential, observed data versus inaccurate self-reported data. One inflection point is where, for instance, the blockchain holds the data and a physician includes a coordination of care plan. For example, if an individual is not sleeping well, the doctor may provide a plan that says to check data of one or more sensors and based on the data showing Y, the individual is to come in for a sleep study. The physician puts in identifiable data and there might be an alert to work with the physician. The individual has the opportunity to make it bidirectional. In this example, the artificial intelligence agent obtains the data, accesses the smart contract and learns of the care plan and executes that plan. For instance, it checks the sensor data and if the analysis shows Y, the artificial intelligence agent recommends a provider to prescribe an action to be taken (e.g., sleep study). Other actions and/or recommendations are possible.

In one or more examples, the artificial intelligence agent may override the care plan. For instance, it may obtain additional data (e.g., other device data and/or exogenous data) and based on an analysis of that data determine that the care plan may be inadequate or inappropriate and thus, communicates the data and analysis to the appropriate health care provider based upon the permissions outlined in the smart contract. Further, it may provide one or more other recommendations. In one example, this occurs if authorized by the smart contract. Further, in one or more examples, if a care plan is to be overridden, this is indicated to one or more entities, as specified in the smart contract. Many variations and possibilities exist.

In one example, based on an individual being alerted of a recommendation (e.g., based on, regardless of, or in lieu of a care plan), the individual has the opportunity to say yes, check with my doctor. Further, in one embodiment, an appointment with the physician is automatically scheduled. In one example, when the intelligent workflow or artificial intelligence agent creates an appointment, the intelligent workflow or artificial intelligence agent pushes the appointment onto the blockchain.

In one or more aspects, a medical professional provides some of their diagnostics against templates for different conditions or use cases: example—if an individual is concerned with a particular virus, then certain behaviors, such as eating odd foods (indicating loss of sense of taste and smell), may be checked. In a further example, if an individual is having a diabetic episode, the glucometer can alert them. Many examples are possible.

In one embodiment, both the physician and device are input to the arrangement, so information to be used is acquired from the physician, device and/or manufacturer of the device. For instance, the data is ingested from the devices and parameters from the manufacturer are provided in set-up. This helps determine whether a particular reading is in a normal range, for instance. As a particular example, the artificial intelligence agent obtains data from selected devices, such as, e.g., a glucometer and insulin pump. The insulin pump provides insulin continuously or based on monitoring via, e.g., a glucometer. If the pump is running low, it sends a message to one or more entities, e.g., the individual and/or a monitoring caregiver.

In accordance with one or more aspects, the data obtained from the devices is not considered in isolation, but other parameters are considered, such as, for instance, the environment of the devices, etc. The data is considered in totality. As an example, the blockchain ingests and stores data, including, for instance, parameters: inputs provided by medical professional(s) and/or device manufacturers; and/or obtained and/or learned data. The blockchain enables the artificial intelligence agent to analyze a combination of data holistically.

In one example, the intelligent workflow (e.g., the artificial intelligence agent) looks holistically at a condition of the person, not just one device. For example, for a diabetic on a continuous positive airway pressure (CPAP) device, the device might lower the individual's breathing—which could be higher and faster because of diabetes, which is not caused by an external factor. The intelligent workflow (e.g., the artificial intelligence agent) determines based, e.g., on analyzing data from one or more devices used by or associated with the individual, manufacturer's data, and/or other data (e.g., medical resources, such as manuals, etc., environmental data, etc.) that additional information is needed and indicates that the individual may need to get their diabetes under control so their CPAP can work. Other examples are possible.

In one or more aspects, the individual authorizes and initiates monitoring, permitting the artificial intelligence agent to notice changes in the individual's condition (e.g., one or more of breathing, heart rate, blood pressure, glucose level, ketones, gait, etc.) and to compare those changes with baseline data to infer the likelihood of a condition, which is brought to the individual's attention with a suggestion of possible action(s). The artificial intelligence agent can combine the risk tolerance of the individual with this data. The artificial intelligence agent can also use the location of the individual to determine what type of alert (e.g., level of detail). For instance, if the individual is at home, show action and/or other confidential health information immediately; in public, provide notification that does not disclose confidential health information; etc. Individualized characteristics are learned and inferred from decisions taken by the individual to build up a persistent risk analysis for the individual that is commensurate with both the observed actions and statements of the individual.

In one or more aspects, the artificial intelligence agent can make suggestions on the coordination of care—e.g., who is to coordinate care, suggestions of which kind of provider the individual needs (e.g., physician, physician assistant, nurse, specialist, nutritionist, food service, care giver, etc.) to augment treatment, remove treatment and/or replace treatment; connects to a coordination of care system; and provides smart contract data sharing. The artificial intelligence agent can provide recommendations and/or optimizations across providers and payers.

In another aspect, a user-authorized and initiated smart contract may be set up to take action on the individual's behalf if the individual is, e.g., incapacitated and cannot make a decision directly to share the data. For example, if an individual's blood sugar drops below a safe threshold, the agent may reach out to medical support for the individual and contact a designated person. As the agent learns more about the individual, the agent may prompt the user with options to adjust the smart contract given a multitude of other data—geolocation, accelerometer, weather, heart rate, etc.

In other embodiments/examples, the artificial intelligence agent may take action to facilitate certain claims for the individual, handle post-vehicle accidents, prepare for upcoming tests/medical procedures, etc. Many variations are possible.

Described herein is an intelligent workflow for a given task, such as healthcare for an individual. Since confidentiality is at the forefront of any medical data and is to be treated with the utmost care, one or more aspects take advantage of the security state of the art. This includes, but is not limited to, using, e.g., secure enclave on personal computing devices, encryption at rest and in transit across secure networks, and/or secure cloud, as examples. One or more aspects provide the ability to share limited, yet meaningful data, with third parties, other agents, etc. in a consultative approach using one or more smart contracts. This allows for time-based sharing of data, that can be revoked after the processing takes place and an answer is returned.

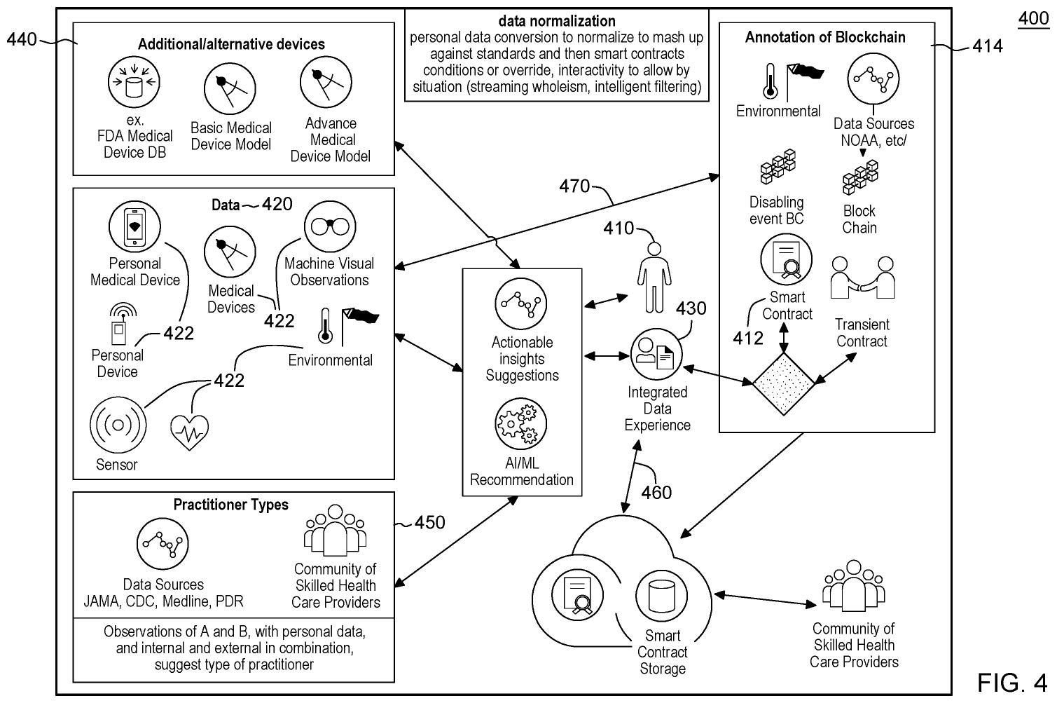

In one or more aspects, the intelligent workflow provides one or more capabilities. Example capabilities are described with respect to FIG. 4; however, an intelligent workflow may include additional, fewer and/or other capabilities in one or more embodiments.

Referring to FIG. 4, in one example, an intelligent workflow 400 places dynamic temporal control of data in the hands of the individual 410. For instance, the patient determines via a smart contract 412 stored on, e.g., a blockchain 414, what data is to be shared with which specific entities, such as health care providers (e.g., physicians, physician assistants, nurses, etc.), service providers (e.g., insurance companies, other type of companies that process claims, food services, care givers, etc.), etc. Further, the data sharing can be modified and/or revoked at any time by the individual. One example of selective sharing includes: consumption of a particular food by an individual was sensed by a device. The individual in the smart contract indicated that the dietary intake may be shared with the individual's physician but not the insurance company. This is indicated in the smart contract written to blockchain and exposed via an electronic health record, as an example.

A further capability of the intelligent workflow includes collecting data 420 from a plurality of sources 422, including, for instance, dedicated sensors/monitors (e.g., medical sensors/monitors, such as glucose monitors, pulse oximeters, heart rate monitors, blood pressure monitors, etc.), cameras (e.g., in-home, smart cameras on homes, buildings, etc.), environmental devices (e.g., smart thermostat, humidity monitors, carbon monoxide detectors, etc.), other equipment and/or devices, etc. An amalgamation of data from a plethora of devices is obtained in a continuously changing environment. An individual's data, temporal and non-temporal status, is collected. Causality is identified due to incorporating data from a multitude of devices including non-purpose built medical and non-medical devices. As an example, available data is pulled in from available devices. In one example, integration middleware across multiple applications that are specific to individual data, but do not aggregate data, is provided. For instance, integration middleware is provided across a glucose monitor app (application), a heart rate measurement app and a smart thermostat app, as examples.

Another capability includes detecting changes 430 in the individual's condition across data from multiple devices. It is designed to provide personalized information and control of the data, including who receives the data, who performs diagnoses, etc., which is retained by the individual. It can keep track of an individual's device information and the individual's medical records. The individual can detect and intercept negative events. The individual's health is monitored seamlessly and non-disruptively and the provision of recommendations is controlled by the individual—user may or may not be provided guidance.

Another capability recommends additional and/or alternate devices 440 to improve data, quantify improvements and connect with commerce engines. This provides individualized data that is not generally available. It provides data that is specific to the individual since it is obtained from monitoring (continuously, periodically, at selected intervals, etc.) the individual. The data from the devices may be converted to an electronic health record. Based on a totality of the data, new devices may be recommended to obtain additional data to fill in gaps to improve recommendations/outcomes for an individual. For example, if a glucose monitoring device is added to an existing data set, the confidence interval for predicting outcomes rises by a selected percentage, such as 20-35%, and a commerce link to purchase that device is provided. Further, as appropriate, an appointment is created with a physician, a prescription is requested, an insurance claim is filed, etc.

Another capability includes providing suggestions 450 to practitioners, services, etc., and/or providing suggestions as to a practitioner and/or service to use. These recommendations are based on a combination of data and conditions and use of resources, such as a Physician Desk Reference and/or other medical resources. As an example, changes in walking stability are measured by monitoring devices and triggers the artificial intelligence agent to recommend a specialist to improve the validity of the data and improve outcomes, and/or recommend a subscription to a data service, meal planning solution, etc. It places dynamic temporal control in the hands of the individual using intelligent workflow. Further, in one example, the control is at the edge.

A further capability includes taking action 460 based on a smart contract if a particular event occurs (e.g., incapacitated). A conditional artificial intelligence agent is created and is informed on whether to take action based on parameters, conditions, internal and exogenous data. If the user is incapacitated, for instance, an appropriate action is taken, such as request assistance given defined parameters. In requesting assistance, the artificial intelligence agent conveys the situation of the individual and the data that triggered the request for help in a way that can quickly and easily be understood. As examples, for an emergency call (e.g., 911), the artificial intelligence agent may use text to speech to provide the data; and for digital healthcare application programming interfaces, it provides the data in digital form. Other examples are possible.

In one example, since the data is combined from several sources, this is an automation to take action only when multiple conditions are met. In one example, instead of placing an emergency call (e.g., calling 911) when the individual's heart rate drops below a certain threshold, the totality of the data, including, for instance, indoor temperature, monitors, such as cameras showing motion or lack thereof, and/or heart rate, etc., are analyzed to determine if an action is warranted, and specifically what action should be taken.

Another capability includes an artificial intelligence driven coordination 470 of care at the edge. Continuous accurate and complete patient data is used that is not dependent on individual self-reporting. In one example, it is at the edge (close to the individual). Recommendations are made, such as which kind of provider is to be used. A connection to coordination of care system is made and a smart contract is provided that defines data sharing. Recommendations and optimizations may be provided across providers and payers.

Although various capabilities of an intelligent workflow are described herein. In other embodiments, an intelligent workflow may include additional, fewer and/or other capabilities. The capabilities described herein are just examples.

Described above is one example of an intelligent workflow. One or more aspects of the process may use machine learning. For instance, machine learning may be used to train the artificial intelligence agent, perform predictive modeling, perform optimization modeling, determine constraints/restrictions, learn from previous data/events, and/or perform other tasks. A system is trained to perform analyses and learn from input data and/or choices made.

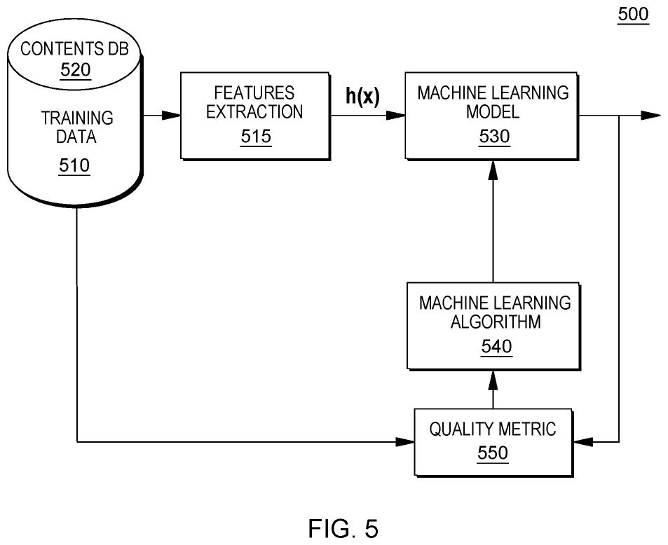

FIG. 5 is one example of a machine learning training system 500 that may be utilized, in one or more aspects, to perform cognitive analyses of various inputs, including data from one or more sources, data repositories and/or other data. Training data utilized to train the model in one or more embodiments of the present invention includes, for instance, data that pertains to devices, including monitors, sensors, environmental devices, etc., data obtained from the devices, data obtained from exogenous sources, actions that have been taken, and/or permissions provided such as in one or more smart contracts, etc. The program code in embodiments of the present invention performs a cognitive analysis to generate one or more training data structures, including algorithms utilized by the program code to predict states of a given event (e.g., a medical event, a medical condition, etc.). Machine learning (ML) solves problems that are not solved with numerical means alone. In this ML-based example, program code extracts various attributes from ML training data 510 (e.g., historical data collected from various data sources relevant to the individual), which may be resident in one or more databases 520 comprising event or task-related data and general data. Attributes 515 are utilized to develop a predictor function, h(x), also referred to as a hypothesis, which the program code utilizes as a machine learning model 530.

In identifying various event states, features, constraints and/or behaviors indicative of states in the ML training data 510, the program code can utilize various techniques to identify attributes in an embodiment of the present invention. Embodiments of the present invention utilize varying techniques to select attributes (elements, patterns, features, constraints, etc.), including but not limited to, diffusion mapping, principal component analysis, recursive feature elimination (a brute force approach to selecting attributes), and/or a Random Forest, to select the attributes related to various events. The program code may utilize a machine learning algorithm 540 to train the machine learning model 530 (e.g., the algorithms utilized by the program code; e.g., an artificial intelligence agent), including providing weights for the conclusions, so that the program code can train the predictor functions that comprise the machine learning model 530. The conclusions may be evaluated by a quality metric 550. By selecting a diverse set of ML training data 510, the program code trains the machine learning model 530 to identify and weight various attributes (e.g., features, patterns, constraints) that correlate to various states of an event.

The model generated by the program code is self-learning as the program code updates the model based on active event feedback, as well as from the feedback received from data related to the event. For example, when the program code determines that there is a condition or event that was not previously predicted by the model, the program code utilizes a learning agent to update the model to reflect the state of the event, in order to improve predictions in the future. Additionally, when the program code determines that a prediction is incorrect, either based on receiving user feedback through an interface or based on monitoring related to the event, the program code updates the model to reflect the inaccuracy of the prediction for the given period of time. Program code comprising a learning agent cognitively analyzes the data deviating from the modeled expectations and adjusts the model to increase the accuracy of the model, moving forward.

In one or more embodiments, program code, executing on one or more processors, utilizes an existing cognitive analysis tool or agent to tune the model, based on data obtained from one or more data sources. One or more embodiments utilize, for instance, an IBM Watson® system as the cognitive agent. In one or more embodiments, the program code interfaces with IBM Watson Application Programming Interfaces (APIs) to perform a cognitive analysis of obtained data. Specifically, in one or more embodiments, certain of the APIs of the IBM Watson API comprise a cognitive agent (e.g., learning agent) that includes one or more programs, including, but not limited to, natural language classifiers, Retrieve and Rank (i.e., a service available through the IBM Watson Developer Cloud™ that can surface the most relevant information from a collection of documents), concepts/visual insights, trade off analytics, document conversion, and/or relationship extraction. In an embodiment, one or more programs analyze the data obtained by the program code across various sources utilizing one or more of a natural language classifier, retrieve and rank APIs, and trade off analytics APIs. The IBM Watson Application Program Interface (API) can also provide audio related API services, in the event that the collected data includes audio, which can be utilized by the program code, including but not limited to natural language processing, text to speech capabilities, and/or translation. IBM Watson® and IBM Watson Developer Cloud™ are registered trademarks or trademarks of International Business Machines Corporation in at least one jurisdiction.

In one or more embodiments, the program code utilizes a neural network to analyze event-related data to generate the model utilized to predict the state of a given event at a given time. Neural networks are a biologically-inspired programming paradigm which enable a computer to learn and solve artificial intelligence problems. This learning is referred to as deep learning, which is a subset of machine learning, an aspect of artificial intelligence, and includes a set of techniques for learning in neural networks. Neural networks, including modular neural networks, are capable of pattern recognition with speed, accuracy, and efficiency, in situations where data sets are multiple and expansive, including across a distributed network, including but not limited to, cloud computing systems. Modern neural networks are non-linear statistical data modeling tools. They are usually used to model complex relationships between inputs and outputs or to identify patterns in data (i.e., neural networks are non-linear statistical data modeling or decision making tools). In general, program code utilizing neural networks can model complex relationships between inputs and outputs and identify patterns in data. Because of the speed and efficiency of neural networks, especially when parsing multiple complex data sets, neural networks and deep learning provide solutions to many problems in multiple source processing, which the program code in one or more embodiments accomplishes when obtaining data and generating a model for predicting states of a given event.

As described above, an intelligent workflow is provided that includes, for instance, using an artificial intelligence agent to obtain device data from a plurality of devices (e.g., including one or more medical devices) for a given individual, as well as data from one or more exogenous sources, including current data relating to one or more medical resources. The artificial intelligence agent analyzes the data obtained from the plurality of devices and the data from the one or more exogenous sources. Based on the analyzing, one or more recommendations are provided. The one or more recommendations include at least one recommendation of a device to use that is different from the plurality of devices to obtain different device data (e.g., additional medical data) for the given individual.

One or more aspects of the present invention are tied to computer technology and facilitate processing within a computer, improving performance thereof. For instance, data storage and retrieval are facilitated using, e.g., smart contracts, improving performance within the computer. In one or more aspects, technical fields of computing, artificial intelligence and healthcare are improved by facilitating the procurement, storage and analysis of vast amounts of data. Processing within a processor, computer system and/or computing environment is improved.

Other aspects, variations and/or embodiments are possible.

The computing environments described herein are only examples of computing environments that can be used. One or more aspects of the present invention may be used with many types of environments. The computing environments provided herein are only examples. Each computing environment is capable of being configured to include one or more aspects of the present invention. For instance, each may be configured to provide intelligent workflow and/or to perform to one or more other aspects of the present invention.

In addition to the above, one or more aspects may be provided, offered, deployed, managed, serviced, etc. by a service manager who offers management of customer environments. For instance, the service manager can create, maintain, support, etc. computer code and/or a computer infrastructure that performs one or more aspects for one or more customers. In return, the service manager may receive payment from the customer under a subscription and/or fee agreement, as examples. Additionally, or alternatively, the service manager may receive payment from the sale of advertising content to one or more third parties.

In one aspect, an application may be deployed for performing one or more embodiments. As one example, the deploying of an application comprises providing computer infrastructure operable to perform one or more embodiments.

As a further aspect, a computing infrastructure may be deployed comprising integrating computer readable code into a computing system, in which the code in combination with the computing system is capable of performing one or more embodiments.

As yet a further aspect, a process for integrating computing infrastructure comprising integrating computer readable code into a computer system may be provided. The computer system comprises a computer readable medium, in which the computer medium comprises one or more embodiments. The code in combination with the computer system is capable of performing one or more embodiments.

Although various embodiments are described above, these are only examples. For example, other data sources and/or devices may be used, other conditions may be considered and/or other recommendations may be provided. Many variations are possible.

Various aspects and embodiments are described herein. Further, many variations are possible without departing from a spirit of aspects of the present invention. It should be noted that, unless otherwise inconsistent, each aspect or feature described and/or claimed herein, and variants thereof, may be combinable with any other aspect or feature.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method of dynamic processing within a computing environment, the computer-implemented method comprising:
automatically obtaining device data from one or more devices for an individual, the one or more devices including one or more medical monitoring devices;
obtaining data from one or more exogenous sources, the data including current data relating to one or more medical resources;
analyzing, using an artificial intelligence agent, the device data obtained from the one or more devices and the data from the one or more exogenous sources; and
providing one or more recommendations, based on the analyzing, the one or more recommendations including an indication of a device to use that is different from the one or more devices to obtain different device data for the individual.

2. The computer-implemented method of claim 1, wherein the analyzing includes obtaining the data from the one or more exogenous sources at selected intervals to obtain the current data relating to the one or more medical resources.

3. The computer-implemented method of claim 1, wherein the analyzing includes comparing the device data to a baseline for the individual to determine that a health-related change has occurred for the individual, and wherein the one or more recommendations are based on the health-related change.

4. The computer-implemented method of claim 1, further comprising providing information of where to obtain the device to use that is different from the one or more devices.

5. The computer-implemented method of claim 1, further comprising initiating performance of one or more actions based on the analyzing, the one or more actions including filing a claim with a health insurance company.

6. The computer-implemented method of claim 1, further comprising initiating performance of one or more actions based on the analyzing, the one or more actions including making an appointment for the individual with a health professional.

7. The computer-implemented method of claim 1, further comprising accessing a smart contract established by the individual to be used in determining the one or more recommendations.

8. The computer-implemented method of claim 7, further comprising:
determining that the individual is incapacitated;
checking based on determining that the individual is incapacitated the smart contract to determine how to proceed; and
alerting at least one entity of the incapacity of the individual based on the smart contract indicating that the at least one entity is to be alerted based on the individual being incapacitated.

9. The computer-implemented method of claim 7, wherein the smart contract explicitly specifies one or more entities to obtain information relating to the individual and one or more types of information to be obtained by the one or more entities.

10. The computer-implemented method of claim 7, wherein the smart contract is stored using a security mechanism.

11. The computer-implemented method of claim 1, wherein the one or more recommendations include a specification of one or more providers for the individual.

12. The computer-implemented method of claim 1, wherein the one or more recommendations include a specification of additional data to be obtained for analysis.

13. A computer system for dynamic processing within a computing environment, the computer system comprising:
a memory; and
one or more processors in communication with the memory, wherein the computer system is configured to perform a method, said method comprising:
automatically obtaining device data from one or more devices for an individual, the one or more devices including one or more medical monitoring devices;
obtaining data from one or more exogenous sources, the data including current data relating to one or more medical resources;
analyzing, using an artificial intelligence agent, the device data obtained from the one or more devices and the data from the one or more exogenous sources; and
providing one or more recommendations, based on the analyzing, the one or more recommendations including an indication of a device to use that is different from the one or more devices to obtain different device data for the individual.

14. The computer system of claim 13, wherein the analyzing includes comparing the device data to a baseline for the individual to determine that a health-related change has occurred for the individual, and wherein the one or more recommendations are based on the health-related change.

15. The computer system of claim 13, wherein the method further comprises accessing a smart contract established by the individual to be used in determining the one or more recommendations.

16. The computer system of claim 15, wherein the method further comprises:
determining that the individual is incapacitated;
checking based on determining that the individual is incapacitated the smart contract to determine how to proceed; and
alerting at least one entity of the incapacity of the individual based on the smart contract indicating that the at least one entity is to be alerted based on the individual being incapacitated.

17. A computer program product for dynamic processing within a computing environment, said computer program product comprising:
one or more computer readable storage media and program instructions collectively stored on the one or more computer readable storage media readable by at least one processing circuit to perform a method comprising:
automatically obtaining device data from one or more devices for an individual, the one or more devices including one or more medical monitoring devices;
obtaining data from one or more exogenous sources, the data including current data relating to one or more medical resources;
analyzing, using an artificial intelligence agent, the device data obtained from the one or more devices and the data from the one or more exogenous sources; and
providing one or more recommendations, based on the analyzing, the one or more recommendations including an indication of a device to use that is different from the one or more devices to obtain different device data for the individual.

18. The computer program product of claim 17, wherein the analyzing includes comparing the device data to a baseline for the individual to determine that a health-related change has occurred for the individual, and wherein the one or more recommendations are based on the health-related change.

19. The computer program product of claim 17, wherein the method further comprises accessing a smart contract established by the individual to be used in determining the one or more recommendations.

20. The computer program product of claim 19, wherein the method further comprises:
determining that the individual is incapacitated;
checking based on determining that the individual is incapacitated the smart contract to determine how to proceed; and
alerting at least one entity of the incapacity of the individual based on the smart contract indicating that the at least one entity is to be alerted based on the individual being incapacitated.
